(19) United States
(12) Patent Application Publication
(10) Pub. No.: US 2025/0204850 A1
(43) Pub. Date: Jun. 26, 2025

(54) MEDICAL IMPLANT WITH TISSUE LOCATION MONITORING

(71) Applicant: Cochlear Limited, Macquarie University, NSW (AU)

(72) Inventors: Werner Meskens, Opwijk (BE); Wim Bervoets, Wilrijk (BE); Koen Erik Van den Heuvel, Hove (BE); Kenneth Oplinger, West Pennant Hills (AU); Stefan Josef Mauger, Macleod (AU)

(21) Appl. No.: 18/848,099

(22) PCT Filed: Mar. 16, 2023
(86) PCT No.: PCT/IB2023/052591

§ 371 Date: Sep. 17, 2024

Related U.S. Application Data
(60) Provisional application No. 63/363,544, filed on Apr. 25, 2022.

(51) Int. Cl. A61B 5/00; A61N 1/05; A61N 1/36

(52) U.S. Cl. CPC A61B 5/4818; A61B 5/4836; A61B 5/682; A61N 1/0548; A61N 1/3611; A61N 1/36139

(57) ABSTRACT

A method includes monitoring a recipient's tongue position while the recipient is asleep. The method further includes (comparing the recipient's tongue position to at least one predetermined threshold position at which the recipient's tongue does not substantially impair breathing of the recipient during sleep. The method further includes, in response to said comparing, selectively generating and applying stimulation signals to the recipient's tongue and/or hypoglossal nerve such that the recipient's tongue moves to a modified position further from substantially impairing breathing of the recipient during sleep than is the recipient's tongue at the at least one predetermined threshold position.

### BACKGROUND

### Field

The present application relates generally to systems and methods for treating sleep-disordered breathing (SDB) conditions, and more specifically, for preventing or minimizing SDB events using stimulation signals from a medical implant.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, a method comprises monitoring a recipient's tongue position while the recipient is asleep. The method further comprises comparing the recipient's tongue position to at least one predetermined threshold position at which the recipient's tongue does not substantially impair breathing of the recipient during sleep. The method further comprises, in response to said comparing, selectively generating and applying stimulation signals to the recipient's tongue and/or hypoglossal nerve such that the recipient's tongue moves to a modified position further from substantially impairing breathing of the recipient during sleep than is the recipient's tongue at the at least one predetermined threshold position.

In another aspect disclosed herein, an apparatus comprises at least one stimulation device configured to generate and apply stimulation signals to a recipient's tongue and/or hypoglossal nerve. The recipient's tongue is responsive to the stimulation signals by moving to be further from substantially impairing respirations by the recipient. The apparatus further comprises at least one sensor configured to monitor positions of the recipient's tongue while the recipient is asleep. The apparatus further comprises control circuitry configured to receive sensor signals from the at least one sensor. The sensor signals are indicative of positions of the recipient's tongue. The control circuitry is further configured to, in response at least in part to the sensor signals, generate and transmit control signals to the at least one stimulation device.

In another aspect disclosed herein, an apparatus comprises at least one electrode configured to stimulate a portion of a recipient's body such that the portion, in response to being stimulated, moves from a first position to a second position. The apparatus further comprises at least one sensor configured to generate sensor signals indicative of positions of the portion. The apparatus further comprises at least one controller configured to respond to the sensor signals by generating and transmitting control signals to the at least one electrode.

### BRIEF DESCRIPTION OF THE DRAWINGS

Implementations are described herein in conjunction with the accompanying drawings, in which:

FIGS. 1A and 1B schematically illustrate an example apparatus in accordance with certain implementations described herein;

FIG. 1C schematically illustrates an example apparatus comprising electrical conduits configured to provide electrical communication among the first circuitry, the second circuitry, and the third circuitry in accordance with certain implementations described herein;

FIG. 1D schematically illustrates a sagittal plane view of a portion of a recipient's skull and tissue with a portion of the first circuitry in accordance with certain implementations described herein;

FIG. 2A schematically illustrates cross-sectional views of three example positions of a recipient's tongue during sleep in accordance with certain implementations described herein;

FIG. 2B schematically illustrates cross-sectional views of two example positions of a recipient's tongue during sleep and the relative positions of three portions of the tongue in these two example positions in accordance with certain implementations described herein;

FIG. 3A schematically illustrates cross-sectional views of two example positions of a recipient's tongue and the at least one sensor in accordance with certain implementations described herein;

FIG. 3B schematically illustrates the two example positions of the at least one sensor of FIG. 3A, without the recipient's anatomy, in accordance with certain implementations described herein;

FIG. 4 schematically illustrates an example sensor that is part of an appliance configured to be disposed within the recipient's mouth in accordance with certain implementations described herein; and

FIGS. 5A and 5B are flow diagrams of two example methods in accordance with certain implementations described herein.

### DETAILED DESCRIPTION

Certain implementations described herein provide a muscle and/or nerve stimulation system configured to trigger stimulations based on the position of a portion of the recipient's anatomy. For example, for a recipient susceptible to obstructive sleep apnea, the system can be configured to sense a position of the tongue while the recipient is asleep and to stimulate the tongue and/or hypoglossal nerve, in response to sensor signals indicative of the tongue position, to move the tongue forward (e.g., away from a position at which the tongue blocks the recipient's airway) before an airway blockage occurs. The system can include one or more devices (e.g., ferromagnetic elements; RFID tags) on or implanted in the tongue and one or more sensors (e.g., external or implanted) configured to generate signals indicative of the relative positions of the one or more devices relative to the one or more sensors. The system can include one or more sensors positioned within the mouth, throat, and/or nose and configured to detect the tongue position (e.g., directly or indirectly via impedance changes, pressure changes, acoustic resonance changes).

The teachings detailed herein are applicable, in at least some implementations, to any type of implantable medical device (e.g., implantable stimulation system; implantable medicament administration system) comprising a first portion on or implanted within the recipient's body and configured to provide stimulation signals and/or medicament dosages to the recipient's body and a second portion (e.g., sensor on or implanted within the recipient or external to the recipient's body) configured to provide control signals to the first portion. For example, the implantable medical device can comprise a neurostimulation system or a muscle stimulation system. In other implementations, in response to detection of an imminent obstructive sleep apnea condition, the device can generate control signals configured to control other external devices (e.g., continuous positive airway pressure or CPAP device being used by the recipient). Implementations can include any type of medical device that can utilize the teachings detailed herein and/or variations thereof.

Merely for ease of description, apparatus and methods disclosed herein are primarily described with reference to an illustrative medical treatment device, namely an implantable treatment system configured to treat sleep-disordered breathing (SDB) conditions, for example, obstructive sleep apnea (OSA) conditions. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical treatment devices beyond those configured to treat sleep-related conditions. For example, apparatus and methods disclosed herein and/or variations thereof may also be used with one or more of the following: vestibular devices (e.g., vestibular implants); sensory prostheses; auditory devices (e.g., bionic ears); auditory prostheses (e.g., cochlear implants); visual devices (e.g., bionic eyes); visual prostheses (e.g., retinal implants); sensors; cardiac pacemakers; drug delivery systems; defibrillators; functional electrical stimulation devices; catheters; brain implants; respiration treatment devices (e.g., devices for treating difficulties with breathing, either while awake or during sleep); seizure devices (e.g., devices for monitoring and/or treating epileptic events); electroporation; pain relief devices; swallowing treatment devices (e.g., devices for treating difficulties with the hyoglossus and/or thyrohyoid muscles); dysphagia treatment devices; devices for treating dry mouth (e.g., xerostomia or hyposalivation), devices for treating excessive or absence of muscle movement due to stroke, Parkinson's disease, or other brain disorders (e.g., preventing “swallowing” of the tongue during an epileptic seizure), devices for treating hypertension (e.g., by stimulating the carotid sinus barosensory system); etc.

Obstructive sleep apnea (OSA), an example of a sleep-disordered breathing (SDB) disorder, is a widespread problem affecting adults in which a person's breathing airways are obstructed during sleep due to loss of tonus of the musculature surrounding the upper airways which results in tissues, either partially or completely, blocking the airways. Such blockage can alter or even stop the person's breathing (e.g., for 20-40 seconds or longer), resulting in snoring, hypoxemia, and/or hypoxia. The discomfort resulting from the stoppage of breathing can partially or fully arouse the person from sleep, upon which the tonus of the surrounding musculature increases, thereby reducing the blockage of the airways by the tissues and allowing the person to resume breathing and to return to sleep. This cycle typically repeats itself throughout the night, sometimes without the person realizing it, and the resultant inadequate sleep can be severe (e.g., disease progression; day-to-day quality of life of the individual; cost to society), leading to poorer quality of life, memory dysfunction, and a higher prevalence of developing or quickening the progression of other diseases.

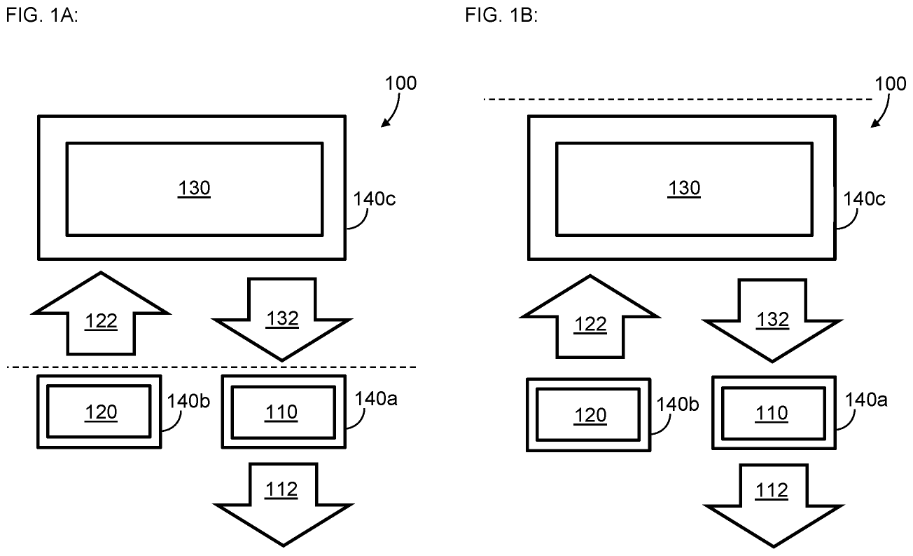

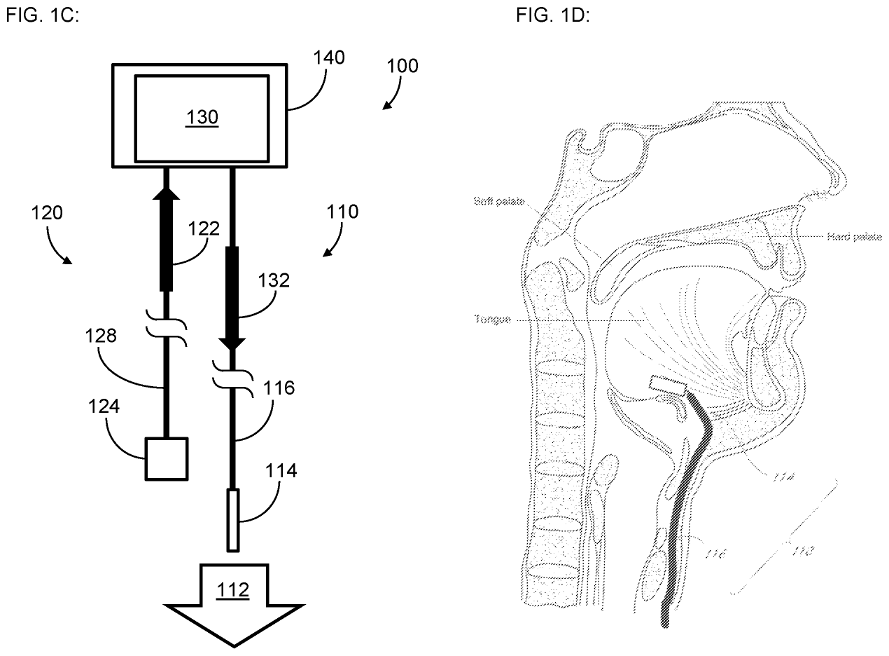

FIGS. 1A and 1B schematically illustrate an example apparatus 100 in accordance with certain implementations described herein. The apparatus 100 comprises first circuitry 110 (e.g., stimulation circuitry; stimulation device) configured to be positioned (e.g., implanted) on or within a recipient's body and is configured to apply stimulation signals 112 to the recipient's body (e.g., the recipient's tongue and/or hypoglossal nerve). The apparatus 100 further comprises second circuitry 120 (e.g., sensor circuitry; at least one sensor) configured to monitor positions of the recipient's tissue (e.g., tongue) while the recipient is asleep. For example, the second circuitry 120 can be on or implanted within the recipient's body and can be configured to generate sensor signals 122 indicative of the position of the recipient's tissue during a sleep session of the recipient. The apparatus 100 further comprises third circuitry 130 (e.g., control circuitry) configured to receive the sensor signals 122 indicative of the position of the recipient's tissue from the first circuitry 110 and, in response at least in part to the sensor signals 122, generate and transmit control signals 132 to the first circuitry 110. For example, the control signals 132 can be indicative of a stimulation signal profile indicative of stimulation signals 112 to be applied to the recipient's tongue and/or hypoglossal nerve to prevent at least one sleep-disordered breathing (SDB) event during the sleep session of the recipient. While certain implementations are described herein as having the third circuitry 130 as a separate component from the first circuitry 110 and the second circuitry 120, in certain implementations, the third circuitry 130 can be integral with the first circuitry 110 and/or integral with the second circuitry 120.

In certain implementations, the apparatus 100 further comprises at least one housing 140 containing (e.g., hermetically sealing within) at least one of the first circuitry 110, the second circuitry 120 and the third circuitry 130. For example, as schematically illustrated in FIG. 1A, the at least one housing 140 comprises a first housing 140a containing at least a portion of the first circuitry 110, a second housing 140b containing at least a portion of the second circuitry 120, and a third housing 140c containing at least a portion of the third circuitry 130. The first and second housings 140a, 140b can be configured to be on or implanted within the recipient's body (e.g., subcutaneously, denoted in FIG. 1A as being below the dashed line), and can be integral with one another (e.g., portions of a common housing) or can be separate from one another. In certain implementations, portions of the at least one housing 140 that are configured to be on or implanted within the recipient's body comprise at least one biocompatible material (e.g., polymer; PEEK; silicone; titanium; titanium alloy; ceramic).

In certain implementations, the at least one housing 140 comprises an implantable portion containing at least a portion of the third circuitry 130 and/or an external portion containing at least a portion of the third circuitry 130. For example, as schematically illustrated by FIG. 1A, the third housing 140c can be external to the recipient's body (denoted in FIG. 1A as being above the dashed line) (e.g., the third housing 140c positioned on an opposite side of the recipient's skin from the implantable first housing 140a and/or second housing 140b). In another example, as schematically illustrated by FIG. 1B, the third housing 140c can be configured to be on or implanted within the recipient's body (denoted in FIG. 1B as being below the dashed line), and can be separate from one or both of the first and second housings 140a, 140b or can be integral with one or both of the first and second housings 140a, 140b (e.g., portions of a common housing). For another example, an external portion of the third circuitry 130 can be within an external housing and an implantable portion of the third circuitry 130 can be within an implantable housing. For another example, at least a portion of the first circuitry 110 and/or the second circuitry 120 can be external to the recipient's body. While FIGS. 1A and 1B schematically illustrate the first circuitry 110 and the second circuitry 120 completely within the recipient's body, in other examples, at least a portion of the first circuitry 110 and/or the second circuitry 120 is within the recipient's body and another portion of the first circuitry 110 and/or the second circuitry 120 is outside (e.g., on) the recipient's body.

In certain implementations, the at least one housing 140 also contains a power source (not shown) of the apparatus 100, for example, a rechargeable battery and/or an antenna (e.g., wire coil comprising multiple turns of electrically insulated single-strand or multi-strand platinum or gold wire) of a transcutaneous energy transfer link configured to wirelessly receive power from a device external to the recipient's body. During operation of the apparatus 100, the power stored by the rechargeable battery can be distributed to the various other implanted components as needed. In certain implementations, the at least one housing 140 further comprises communication circuitry (not shown) configured to receive signals from and/or transmit signals to a device external to the recipient's body. For example, the communication circuitry can comprise at least one signal transceiver having at least one antenna (e.g., wire coil comprising multiple turns of electrically insulated single-strand or multi-strand platinum or gold wire) that is part of an inductive radio frequency (RF) communication link and is configured to receive control information wirelessly transmitted from the external device and/or to wirelessly transmit status information to the external device.

In certain implementations, the first circuitry 110 comprises at least a portion of an implantable stimulation device and comprises at least one stimulation element 114 (e.g., electrical electrode; electrical contact; optical emitter; optical contact) configured to be positioned (e.g., implanted) on or within the recipient's body and to apply the stimulation signals 112 to a portion of the tongue of the recipient and/or to a portion of a hypoglossal nerve of the recipient. The first circuitry 110 of certain implementations comprises a muscle stimulation device (see, e.g., U.S. Pat. No. 8,892,205) in which the at least one stimulation element 114 is implanted within the recipient's tongue and/or chin and is configured to apply the stimulation signals 112 (e.g., electrical stimulation signals; optical stimulation signals) to a portion of the recipient's tongue. In certain other implementations, the first circuitry 110 comprises a neuromodulation device (e.g., a hypoglossal nerve stimulation device, see, e.g., U.S. Pat. Nos. 9,415,215 and 9,943,686) in which the at least one stimulation element 114 (e.g., an electrode cuff surrounding a portion of a hypoglossal nerve of the recipient) is configured to apply the stimulation signals 112 to a portion of the hypoglossal nerve. The stimulation signals 112 generated by the first circuitry 110 are configured to evoke movement of the recipient's tongue (e.g., moving to be further from substantially impairing breathing of the recipient).

FIG. 1C schematically illustrates an example apparatus 100 comprising first circuitry 110, second circuitry 120, and third circuitry 130 in accordance with certain implementations described herein. FIG. 1D schematically illustrates a sagittal plane view of a portion of a recipient's skull and tissue with a portion of the first circuitry 110 in accordance with certain implementations described herein. In certain implementations, as schematically illustrated by FIG. 1C, the first circuitry 110 has a proximal end connected to the third circuitry 130 (e.g., connected to an implantable portion of the at least one housing 140), and a distal end comprising the at least one stimulation element 114. For example, the first circuitry 110 can comprise a signal conduit 116 (e.g., wire; cable) configured to transmit the control signals 132 from the third circuitry 130 to the at least one stimulation element 114. In certain other implementations, the signal conduit 116 is absent and the third circuitry 130 is in wireless communication with the at least one stimulation element 114. For example, the control signals 132 can be wirelessly transmitted from the third circuitry 130 to the at least one stimulation element 114 (e.g., transmitted by radio frequency signals from an antenna of the third circuitry 130 to an antenna of the first circuitry 110). In certain implementations, at least a portion of the first circuitry 110 is hermetically sealed within a biocompatible housing 140a (e.g., a stimulator unit) separate from the housing 140c comprising the third circuitry 130.

While FIG. 1D schematically illustrates the first circuitry 110 as being implanted within the recipient's body, in certain other implementations, the first circuitry 110 is configured to be worn over the recipient's skin (e.g., on or outside the skin). For example, the first circuitry 110 can comprise a patch configured to be placed under the recipient's chin and at least one electrode configured to contact the recipient's skin. The at least one electrode can be configured to apply electrical stimulation signals to the hypoglossal nerve (e.g., using stimulation levels sufficiently large for excitation through the tissue between the electrode and the hypoglossal nerve). While FIG. 1D schematically illustrates the at least one stimulation element 114 in or near the tongue, in certain other implementations, the at least one stimulation element 114 is in proximity to a portion of the hypoglossal nerve spaced from the tongue. While FIG. 1D schematically illustrates the signal conduit 116 extending between the tongue through the neck (e.g., to other circuitry in the chest and/or lung), in certain other implementations, the signal conduit 116 is shorter and connects the at least one stimulation element 114 to other circuitry closer to the tongue and/or chin.

In certain implementations, the second circuitry 120 comprises at least one sensor 124 configured to be responsive to a position of the recipient's tongue by generating the sensor signals 122 (e.g., comprising information indicative of the position of the tongue) received by the third circuitry 130 to trigger the first circuitry 110. As schematically illustrated by FIG. 1C, the third circuitry 130 is configured to receive the sensor signals 122 from the at least one sensor 124 and to extract the position information from the sensor signals 122. In certain implementations, the second circuitry 120 is configured to continuously stream the sensor signals 122 to the third circuitry 130 during the sleep session.

In certain implementations, the second circuitry 120 has a proximal end connected to the third circuitry 130 (e.g., connected to an implantable portion of the at least one housing 140), and a distal end comprising the at least one sensor 124. For example, as schematically illustrated by FIG. 1C, the second circuitry 120 can comprise a signal conduit 128 (e.g., wire; cable) configured to transmit the sensor signals 122 from the at least one sensor 124 to the third circuitry 130. In certain other implementations, the signal conduit 128 is absent and the third circuitry 130 is in wireless communication with the at least one sensor 124. For example, the sensor signals 122 can be wirelessly transmitted from the at least one sensor 124 to the third circuitry 130 (e.g., transmitted by radio frequency signals from an antenna of the at least one sensor 124 to an antenna of the third circuitry 130).

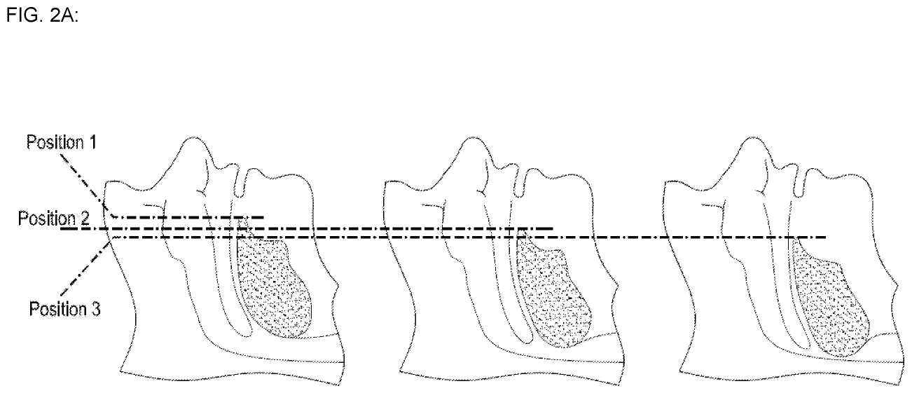

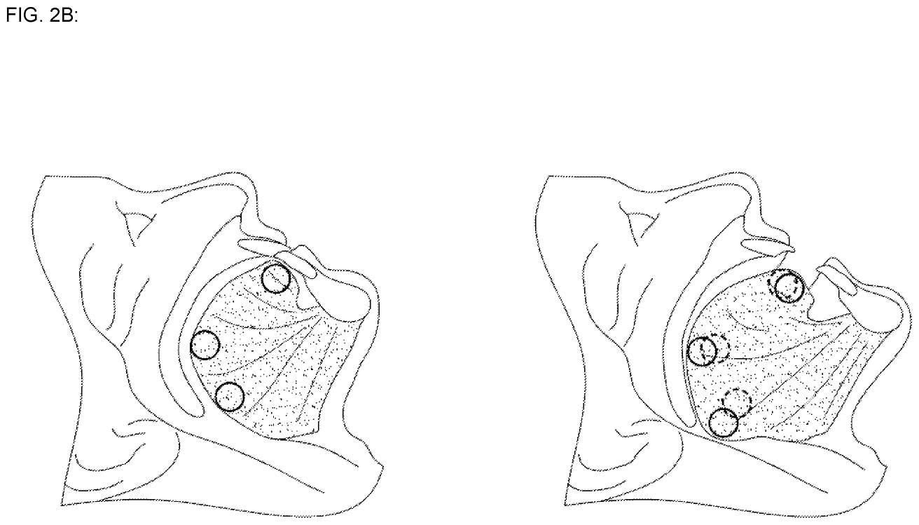

FIG. 2A schematically illustrates cross-sectional views of three example positions of a recipient's tongue during sleep in accordance with certain implementations described herein. In the left-most view of FIG. 2A, the recipient is experiencing nominal sleep conditions with normal breathing (e.g., respiration) through an open airway, and the tip of the recipient's tongue is at a first position and an SDB event is not occurring. In the center view of FIG. 2A, the recipient is snoring while breathing (e.g., respiration) through an airway partially blocked by the tongue (e.g., the tongue is not substantially impairing breathing of the recipient), and the tip of the recipient's tongue is at a second position closer to the throat than is the first position (e.g., farther back within the mouth than is the first position). With the tongue at the second position, an SDB event is not yet occurring but the likelihood of an SDB event occurring is higher than for the first position (e.g., an SDB event is imminent). In the right-most view of FIG. 2A, the recipient's tongue is substantially (e.g., fully) blocking the airway (e.g., substantially impairing breathing or respiration of the recipient), and the tip of the recipient's tongue is at a third position closer to the throat than is the second position (e.g., farther back within the mouth than is the second position). With the tongue at the third position, an SDB event (e.g., obstructive sleep apnea; substantially impaired breathing or respiration) is occurring.

FIG. 2B schematically illustrates cross-sectional views of two example positions of a recipient's tongue during sleep and the relative positions of three portions of the tongue in these two example positions in accordance with certain implementations described herein. In the left view of FIG. 2B, the recipient is experiencing nominal sleep conditions with normal breathing through an open airway (e.g., non-SDB sleep), and the three portions (e.g., the tip, the center, and the back of the tongue; denoted by solid circles) are at three positions indicative of non-SDB sleep. In the right view of FIG. 2B, the recipient's tongue is substantially (e.g., fully) blocking the airway (e.g., substantially impairing breathing of the recipient), and the three portions (denoted by solid circles) of the recipient's tongue are at three positions indicative of an existing and/or imminent SDB event, these three positions different from the three positions (denoted by the dashed circles) indicative of non-SDB sleep. With the tongue at the third position, an SDB event (e.g., obstructive sleep apnea) is occurring.

In certain implementations, the at least one sensor 124 is responsive to a position of a device on or within the recipient's tongue. FIGS. 2A and 2B show that the positions of various portions of the recipient's tongue (e.g., the tip, the center, and/or the back of the recipient's tongue) are correlated with the sleep conditions experienced by the recipient (e.g., whether an SDB event is not imminent, is imminent, or is occurring). In certain implementations, the at least one sensor 124 of the second circuitry 120 is configured to monitor (e.g., detect; measure) the positions of one or more portions of the recipient's tongue while the recipient is asleep. The sensor signals 122 from the at least one sensor 124 can provide information regarding the positions of the one or more portions of the tongue to the third circuitry 130.

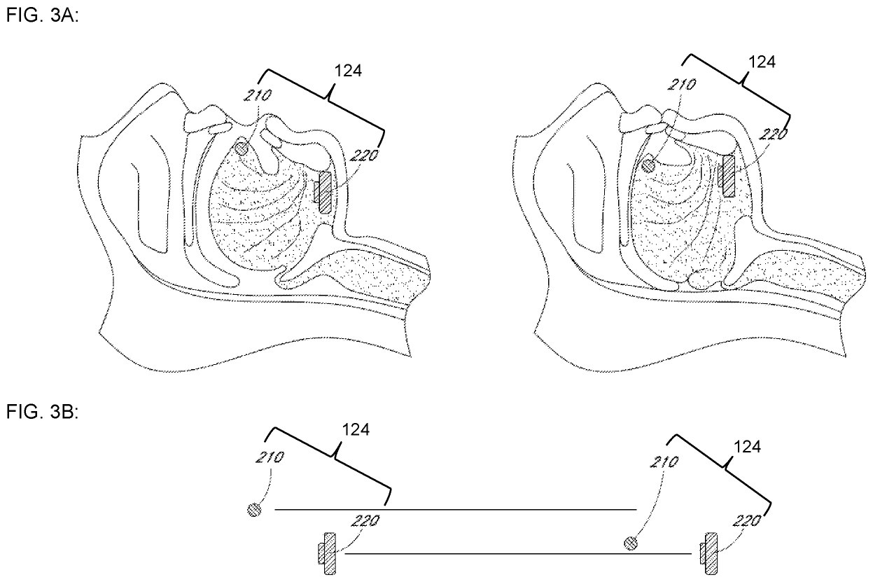

FIG. 3A schematically illustrates cross-sectional views of two example positions of a recipient's tongue and the at least one sensor 124 in accordance with certain implementations described herein. FIG. 3B schematically illustrates the two example positions of the at least one sensor 124 of FIG. 3A, without the recipient's anatomy, in accordance with certain implementations described herein. As shown in FIGS. 3A-3B, the at least one sensor 124 comprises at least one first device 210 on or within the recipient's tongue (e.g., the tip of the recipient's tongue) and at least one second device 220 (e.g., active sensor; passive sensor) configured to generate the sensor signals 122 in response to a relative position of the at least one first device 210 to the at least one second device 220.

For example, the at least one first device 210 can comprise a ferromagnetic material (e.g., one or magnetic tongue piercings; tubes; needles; pins) in or on the tongue. The at least one second device 220 can comprise a magnetic field sensor (e.g., static magnetic field sensor; Hall sensor; giant magneto-resistive sensor; anisotropic magneto-resistive sensor; capacitive plates; inductive loop; electromagnetic antenna) configured to generate sensor signals 122 indicative of the relative position of the at least one first device 210 to the at least one second device 220. For another example, the at least one first device 210 can comprise at least one radio-frequency identification (RFID) tag and the at least one second device 220 can comprise at least one RFID reader.

In certain implementations, the at least one first device 210 is configured to be implanted on or in the tongue, while in certain other implementations, the at least one first device 210 is configured to be removably affixed to the tongue (e.g., configured to be repeatedly attached to and detached from the recipient's tongue; configured to be removed prior to the recipient undergoing magnetic resonance imaging). For example, the at least one first device 210 can comprise a sleeve configured to be temporarily worn on (e.g., over) the tongue during a sleep session and removed during waking hours. In certain implementations, the at least one second device 220 can be integral with the first circuitry 110 configured to apply the stimulation signals 112 to the recipient's tongue and/or hypoglossal nerve (e.g., an implanted stimulation assembly). In certain other implementations, the at least one second device 220 is separate from the first circuitry 110 and/or the third circuitry 130 (e.g., configured to be worn by the recipient and to be repeatedly removed and replaced). For example, the at least one second device 220 can comprise an appliance (e.g., retainer) configured to be repeatedly inserted within the recipient's mouth and removed from the recipient's mouth (e.g., temporarily located within the mouth during a sleep session and removed during waking hours). For another example, the at least one second device 220 can be worn outside the recipient's mouth. Certain such implementations having temporarily worn first and second devices 210, 220 can provide recipients and medical practitioners (e.g., caregivers) the ability to confirm that the apparatus 100 can provide relieve from SDB events before undergoing implant surgery and/or to identify operational or problematic locations of the first and second devices 210, 220. In certain implementations, the apparatus 100 comprises multiple first devices 210 and the at least one sensor 124 is configured to track the positions of multiple portions of the tongue (e.g., multiple first devices 210 at each of the circled portions of FIG. 2B). The third circuitry 130 can be configured to trigger the stimulation signals 112 upon all of the first devices 210 being at positions indicative of an SDB event or can be configured to trigger the stimulation signals 112 upon at least one of the first devices 210 being at positions indicative of an SDB event.

In certain implementations, the at least one sensor 124 comprises at least one electrode configured to detect physical contact with the recipient's tongue and/or electrical impedance changes due to movement of the recipient's tongue. For example, the at least one sensor 124 can comprise at least one electrode disposed at the back of the throat that is configured to generate signals indicative of physical contact between the recipient's tongue and the at least one electrode (e.g., presence of such physical contact indicative of potential and/or substantive blockage of the airway by the tongue in an SDB event). For another example, the at least one sensor 124 can comprise at least one electrode disposed within the mouth and/or throat (e.g., in or on the front of the mouth held in place by the lips and/or teeth, roof, soft palate, uvula, or back of the throat) that is configured to generate a signal indicative of physical contact between the recipient's tongue and the at least one electrode (e.g., absence of such physical contact indicative of potential and/or substantive blockage of the airway by the tongue in an SDB event). For another example, the at least one sensor 124 can comprise at least one electrode disposed in or on a portion of the tongue, the at least one electrode configured to generate signals indicative of physical contact between the portion of the tongue and other tissue. Depending on the other tissue and/or the portion of the tongue at which the at least one electrode is positioned, the presence or absence of such physical contact can be indicative of potential and/or substantive blockage of the airway by the tongue in an SDB event). Other locations of the at least one electrode are also compatible with certain implementations described herein.

In certain implementations, the at least one sensor 124 comprises at least one ultrasound detector (e.g., microphone) within the recipient's mouth and/or nose, the at least one ultrasound detector configured to detect sound changes and/or acoustic resonance changes within the recipient's mouth and/or nose due to movement of the recipient's tongue. Such changes can be indicative of variations in the airway obstruction level (e.g., indicating that an airway obstruction is imminent or has occurred). Other locations of the at least one ultrasound detector (e.g., within an implant; within an external component) are also compatible with certain implementations described herein. In certain implementations, the apparatus 100 can further comprise an ultrasonic source configured to generate ultrasonic signals to be detected by the at least one ultrasound detector (e.g., in a common housing with the ultrasound detector for a more compact implementation).

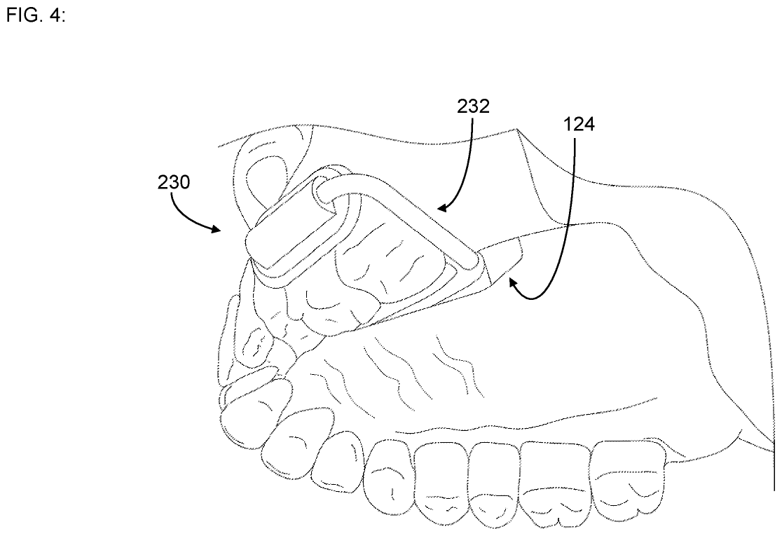

FIG. 4 schematically illustrates an example sensor 124 that is part of an appliance 230 configured to be disposed within the recipient's mouth in accordance with certain implementations described herein. The appliance 230 can be configured to be repeatedly positioned within the recipient's mouth and removed from the recipient's mouth (e.g., temporarily located within the mouth during a sleep session and removed during waking hours). For example, as shown in FIG. 4, the appliance 230 can comprise the sensor 124 and a clamp 232 affixed to the sensor 124, the clamp 232 configured to be temporarily affixed to one or more teeth of the recipient and to hold the sensor 124 in position within the mouth. The sensor 124 can be configured to generate the sensor signals 122 indicative of the position of the tongue. For example, the sensor 124 can comprise an impedance sensor (e.g., responsive to changes of electrical impedance that are dependent on the tongue's position), a pressure or contact sensor (e.g., responsive to whether the sensor is contacted by the tongue or not), and/or an ultrasound generator and detector (e.g., responsive to sound changes and/or acoustic resonance changes within the recipient's mouth and/or nose due to movement of the tongue).

In certain implementations, the third circuitry 130 comprises one or more digital signal processors (DSPs), one or more microcontroller cores, one or more application-specific integrated circuits (ASICs), firmware, software, etc. configured to receive the sensor signals 122 from the second circuitry 120 during the sleep session and in response at least in part to the sensor signals 122, to provide control signals 132 to the first circuitry 110 during the sleep session. For example, the third circuitry 130 can be configured to extract the information indicative of the position of the recipient's tongue during the sleep session from the sensor signals 122, to evaluate the information in real-time, and to determine appropriate control signals 132 to be provided to the first circuitry 110 during the sleep session. The information obtained from the sensor signals 122 regarding the positions of the one or more portions of the tongue can be used by the third circuitry 130 to prevent SDB events by triggering the first circuitry 110 to apply the stimulation signals 112 to the recipient's tongue and/or hypoglossal nerve prior to an SDB event occurring (e.g., when an SDB event is imminent; before the tongue is substantially impairing breathing of the recipient; upon movement of the tongue towards an airway blocking position). In certain implementations, the third circuitry 130 operates in a closed loop, using the sensor signals 122 to selectively trigger stimulation of the tongue and/or hypoglossal nerve upon detection of conditions in which an SDB event is imminent but is not yet occurring. In contrast to systems which periodically stimulate the tongue and/or hypoglossal nerve at regular temporal intervals throughout the sleep session (e.g., open loop operation), by only generating stimulation signals in response to the detected position of one or more portions of the tongue (e.g., closed loop operation), certain implementations described herein can prevent SDB events at reduced power consumption levels and/or while evoking less tongue fatigue (e.g., exhaustion of the tongue muscles such that the tongue muscles become less responsive to the stimulation signals 112).

In certain implementations, the control signals 132 are indicative of a stimulation signal profile that comprises one or more parameters of the stimulation signals 112 to be applied to the recipient's body during the sleep session. In certain implementations, the stimulation signal profile comprises at least one of: a repetition rate of the stimulation signals 112 (e.g., number per unit time; frequency), a burst duration of the stimulation signals 112 (e.g., a time period during which a sequence of stimulation signals 112 is applied), and a temporal amplitude profile of the stimulation signals 112 (e.g., a pulse amplitude as a function of time, which can include the temporal shapes of the individual stimulation signal pulses and/or the temporal shape of an envelope of stimulation signal pulses during a burst of the stimulation signals 112). In certain implementations, the stimulation signal profile comprises at least one parameter of the stimulation therapy, examples of which include but are not limited to: an onset voltage, an offset voltage, and an amplitude of the stimulation signals 112.

In certain implementations, the stimulation signals described by the stimulation signal profile are configured to prevent at least one SDB event (e.g., an OSA breathing stoppage) during the sleep session while preventing at least one disruption of the sleep session by the stimulation signals 112 (e.g., waking the recipient due to the physiological response evoked by the stimulation signals 112, whether perceived or not by the recipient). For example, the stimulation signal profile can be configured such that a first number of SDB events experienced by the recipient during a current sleep session with the stimulation signal profile is lower than a second number of SDB events experienced by the recipient during a previous sleep session with a different stimulation signal profile (e.g., the current and previous sleep sessions having the same duration and other conditions). In addition, the stimulation signal profile can be configured such that a third number of sleep disruptions during a current sleep session with the stimulation signal profile is lower than a fourth number of sleep disruptions during a previous sleep session with a different stimulation signal profile (e.g., the current and previous sleep sessions having the same duration and other conditions).

In certain implementations in which the recipient is wearing a continuous positive airway pressure (CPAP) device during the sleep session, the third circuitry 130 is further configured to, in response at least in part to the sensor signals 122 indicative of the position of the recipient's tongue, provide CPAP control signals to the CPAP device. For example, in response to the sensor signals 122, the third circuitry 130 can generate and provide CPAP control signals that the CPAP device responds to by adjusting the airflow provided to the recipient (e.g., to counteract breathing-based disruptions and to allow the sleep session to continue with less disruption).

In certain implementations, the third circuitry 130 is configured to receive additional information, besides the tissue position information from the sensor signals 122 generated by the second circuitry 120, and to, in response at least in part to the additional information, provide the control signals 132 to the first circuitry 110. For example, the third circuitry 130 can be configured to receive status signals from the first circuitry 110 (e.g., indicative of an operational status of at least one component of the first circuitry 110; record of stimulation signals 112 actually provided). For another example, the third circuitry 130 can be configured to receive (e.g., wired or wirelessly) additional signals from additional sensor circuitry (e.g., from one or more additional sensors that are part of the apparatus 100 or that are separate from the apparatus 100). For example, the additional signals can be indicative of the recipient's physiology during sleep (e.g., eye movements; muscle movements; cardiac activity; blood oxygen levels; respiration); recipient's actions/responses during sleep (e.g., limb/leg/arm/torso movement); recipient-generated sounds (e.g., snoring, coughing, wheezing, labored breathing, or talking) during sleep; ambient environmental condition (e.g., air temperature; bed or body temperature; humidity; air pressure; ambient light intensity) during sleep; parameters of operation of a continuous positive airway pressure (CPAP) device worn by the recipient during sleep (e.g., airflow; breathing pattern).

In certain implementations, the third circuitry 130 is configured to access at least one non-transitory memory device configured to provide information to the third circuitry 130. In certain implementations, the at least one non-transitory memory device is configured to store information obtained from the responses of at least one sensor 124 and/or from the recipient. Examples of the at least one non-transitory memory device can include but are not limited to: random-access memory (RAM) integrated circuit; flash memory; hard-disk drive; other tangible data storage device. In certain implementations, the at least one non-transitory memory device is an integral component of the third circuitry 130, while in certain other implementations, the at least one non-transitory memory device is a component of a smartphone, smart tablet, smart watch, electronic diary, or remote device operated by the recipient. In certain implementations, the at least one non-transitory memory device is configured to be accessed by the third circuitry 130 via the internet (e.g., the at least one non-transitory memory device is part of the cloud).

In certain implementations, the apparatus 100 is configured to be self-fitting to the recipient, trainable (e.g., self-learning), and/or to optimize the sleep quality for the recipient. For example, the third circuitry 130 can be configured to access (e.g., from the at least one non-transitory memory device), during a sleep session of the recipient, subjective information (e.g., at least one post-sleep session evaluation by the recipient) indicative of sleep quality of at least one prior sleep session of the recipient. The third circuitry 130 can be further configured to access (e.g., from the at least one non-transitory memory device), during the sleep session, at least one prior stimulation signal profile indicative of the stimulation signals 112 applied by the first circuitry 110 during the at least one prior sleep session of the recipient. The third circuitry 130 can be further configured to, in response at least in part to the subjective information and the at least one prior stimulation signal profile, provide the control signals 132 to the first circuitry 110 during the sleep session. The control signals 132 can be configured such that the sleep session has fewer and/or less severe SDB events than does the at least one prior sleep session.

In certain implementations, the stimulation signals 112 applied during the sleep session have at least one stimulation parameter (e.g., repetition rate; burst duration; temporal amplitude profile) different from that of the stimulation signals applied during the at least one prior sleep session. For example, the at least one stimulation parameter can be automatically adjusted or titrated to arrive at an effective value that reduces (e.g., minimizes) the number and/or severity of SDB events and/or sleep disruptions induced by the treatment. The third circuitry 130 of certain implementations is further configured to provide the stimulation signal profile of the sleep session to be stored (e.g., by the non-transitory memory device) to be accessible by the third circuitry 130 during subsequent sleep sessions.

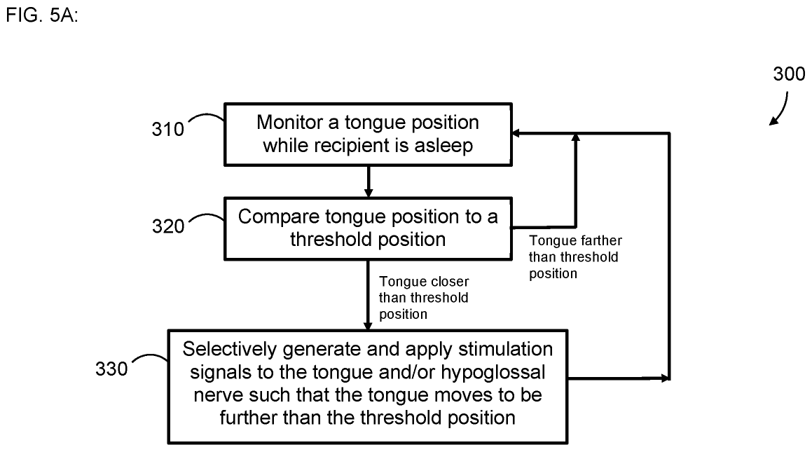

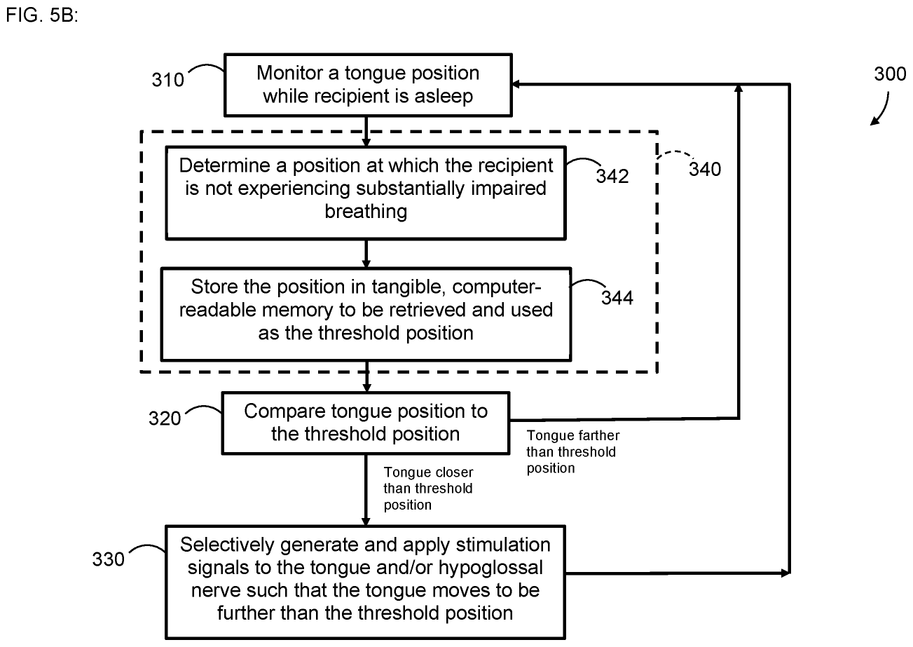

FIGS. 5A and 5B are flow diagrams of two example methods 300 in accordance with certain implementations described herein. The method 300 of certain implementations can utilize closed loop control to generate and apply the stimulation signals 112 in response to the position of the recipient's tongue. While the method 300 is described by referring to some of the structures of the example apparatus 100 and anatomy of FIGS. 1A-1D, 2A-2B, 3A-3B, and 4, other apparatus and systems with other configurations of components and/or used in conjunction with other anatomy can also be used to perform the method 300 in accordance with certain implementations described herein.

In an operational block 310, the method 300 comprises monitoring a recipient's tongue position while the recipient is asleep. For example, the method 300 can be performed using the apparatus 100 described herein to treat obstructive sleep apnea.

In an operational block 320, the method 300 further comprises comparing the recipient's tongue position to at least one predetermined threshold position at which the recipient's tongue does not substantially impair breathing of the recipient during sleep. For example, the third circuitry 130 can compare the recipient's tongue position, determined in response to the sensor signals 122 from the second circuitry 120, to at least one predetermined threshold position (e.g., retrieved from at least one non-transitory memory device) at which the recipient's tongue is not substantially (e.g., fully) blocking the airway. In certain implementations, the at least one predetermined threshold position can be offset (e.g., by a predetermined amount) from a position at which breathing begins to be substantially impaired.

In an operational block 330, the method 300 further comprises, in response to said comparing, selectively generating and applying stimulation signals to the recipient's tongue and/or hypoglossal nerve such that the recipient's tongue moves to a modified position further from substantially impairing breathing of the recipient during sleep than is the recipient's tongue at the at least one predetermined threshold position. For example, as shown in FIGS. 5A and 5B, said selectively generating and applying the stimulation signals can be performed only when the recipient's tongue is in a range of positions closer to substantially impairing breathing of the recipient than is the at least one predetermined threshold position (e.g., stimulating the tongue only when the tongue is not in an acceptable range of positions). The at least one predetermined threshold positions can define a window (e.g., an alert window or zone) comprising a set of tongue positions at which stimulation of the tongue is to be performed but before a substantial airway obstruction occurs. Said selectively generating and applying the stimulation signals can be performed using a stimulation device (e.g., first circuitry 110) that in certain implementations is on or implanted within the recipient's body, or in certain other implementations is outside the recipient's body.

In certain implementations, as shown in FIG. 5B, in an operational block 340, the method 300 further comprises setting the at least one predetermined threshold position. As shown in FIG. 5B, in an operational block 342, the method 300 can comprise, in response to said monitoring, determining at least one position of the recipient's tongue at which the recipient is not experiencing substantially impaired breathing (e.g., a position that is offset from a position at which breathing is detected to have begun to be substantially impaired), and in an operational block 344, the method 300 can comprise storing the at least one position in tangible, computer-readable memory to be retrieved and used as the at least one predetermined threshold position.

For example, while the recipient is asleep, the third circuitry 130 can receive the sensor signals 122 indicative of the current tongue position and additional signals indicative of whether the recipient is experiencing substantially impaired breathing while the tongue is at the current tongue position. If the recipient is not experiencing substantially impaired breathing (e.g., not experiencing an SDB event), the tongue position value can be stored in a buffer. As the monitoring continues and the tongue moving while the recipient is asleep, upon the additional signals indicating that the recipient is experiencing substantially impaired breathing (e.g., experiencing an SDB event), the latest tongue position value stored in the buffer can be used to calculate a threshold position. For example, the threshold position can be the latest tongue position value stored in the buffer or can be a tongue position value that is further (e.g., by a predetermined amount or percentage) from the tongue position value at which the recipient begins experiencing substantially impaired breathing.

In certain implementations, said setting the at least one predetermined threshold position can be performed automatically by a medical device (e.g., sleep apnea treatment system) configured to perform said monitoring, said comparing, and said generating and applying the stimulation signals. For example, the apparatus 100 can be configured to be self-fitting (e.g., self-learning) such that during an initial sleep session, the stimulation signals 112 are generated and applied upon the tongue reaching an initial (e.g., default) threshold position and the threshold position used to trigger the stimulation signals 112 can be adjusted (e.g., closer to initiating an SDB event; farther from initiating an SDB event) later during the initial sleep session and/or during subsequent sleep sessions to tune the threshold position to a value at which SDB events are sufficiently avoided while using a sufficiently reduced number of stimulations.

In certain other implementations, said setting the at least one predetermined threshold position can be performed during a fitting (e.g., calibration) session in which an acceptable range of tongue positions and/or an unacceptable (e.g., problematic) range of tongue positions are determined based on the sensor signals 122 indicative of the detected tongue positions and the additional signals indicative of whether the recipient is experiencing substantially impaired breathing. For example, the fitting session can be performed at a sleep clinic to externally measure when the recipient has substantially impaired breathing and simultaneously monitor the tongue position. In certain implementations, machine learning algorithms can be used to assist with identifying the acceptable and/or unacceptable ranges of tongue positions. Said setting the at least one predetermined threshold position can entail refitting sessions, depending on whether the locations of the at least one sensor 124 changes and to the extent of such changes (e.g., drifts of implants in the tongue through mechanical movement of the tongue; other natural processes).

In certain implementations, the method 300 is configured to set the applied stimulation level automatically based on the measured tongue position. For example, the applied stimulation level can be set at an initial level and then incrementally increased and/or decreased to find a level at which the tongue moves into a position at which the recipient is not experiencing substantially impaired breathing while not being excessively disruptive to the recipient's sleep. This adjustment of the applied stimulation level can be performed once, (e.g., in the first week of use) or can be performed at selected or periodic intervals (e.g., multiple times during years of use). Certain such implementations can provide easier use (e.g., less clinical work) and/or lower power consumption (e.g., using lower stimulation levels to achieve efficacious effect). Certain such implementations can also avoid manual retuning if recipient's anatomy changes over time and/or if the stimulation electrodes change position.

In certain implementations, the method 300 can further comprise using additional sensors to generate additional sensor signals which are used (e.g., by the third circuitry 130) to supplement the sensor signals 122 comprising information indicative of the tongue position. For example, an implantable microphone can be used to detect body noise changes (e.g., indicative of an airway obstruction).

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” “less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. A method comprising:
monitoring a recipient's tongue position while the recipient is asleep;
comparing the recipient's tongue position to at least one predetermined threshold position at which the recipient's tongue does not substantially impair breathing of the recipient during sleep; and
in response to said comparing, selectively generating and applying stimulation signals to the recipient's tongue and/or hypoglossal nerve such that the recipient's tongue moves to a modified position further from substantially impairing breathing of the recipient during sleep than is the recipient's tongue at the at least one predetermined threshold position.

2. The method of claim 1, wherein the method is performed to treat obstructive sleep apnea.

3. The method of claim 1, further comprising setting the at least one predetermined threshold position by:
in response to said monitoring, determining at least one detected position of the recipient's tongue at which the recipient is not experiencing substantially impaired breathing; and
storing the at least one detected position in tangible, computer-readable memory to be retrieved and used as the at least one predetermined threshold position.

4. The method of claim 3, wherein said setting the at least one predetermined threshold position is performed automatically by a device configured to perform said monitoring, said comparing, and said generating and applying stimulation signals.

5. The method of claim 1, wherein said selectively generating and applying stimulation signals is performed only when the recipient's tongue is in a range of positions closer to substantially impairing breathing of the recipient than is the at least one predetermined threshold position.

6. The method of claim 1, wherein said selectively generating and applying stimulation signals is performed using a stimulation device on or implanted within the recipient's body.

7. The method of claim 1, wherein said selectively generating and applying stimulation signals is performed using a stimulation device outside the recipient's body.

8. An apparatus comprising:
at least one stimulation device configured to generate and apply stimulation signals to a recipient's tongue and/or hypoglossal nerve, the recipient's tongue responsive to the stimulation signals by moving to be further from substantially impairing respirations by the recipient;
at least one sensor configured to monitor positions of the recipient's tongue while the recipient is asleep; and
control circuitry configured to:
receive sensor signals from the at least one sensor, the sensor signals indicative of positions of the recipient's tongue; and
in response at least in part to the sensor signals, generate and transmit control signals to the at least one stimulation device.

9. The apparatus of claim 8, wherein the control circuitry is in wireless communication with the at least one sensor and/or the at least one stimulation device.

10. The apparatus of claim 8, wherein the at least one sensor is configured to be responsive to a position of a device on or within the recipient's tongue.

11. The apparatus of claim 10, wherein the device comprises a ferromagnetic material and the sensor comprises a magnetic field sensor.

12. The apparatus of claim 10, wherein the device comprises at least one radio-frequency identification (RFID) tag and the sensor comprises at least one RFID reader.

13. The apparatus of claim 10, wherein the device is configured to be repeatedly attached to the recipient's tongue and detached from the recipient's tongue.

14. The apparatus of claim 8, wherein the at least one sensor is configured to be disposed in the recipient's body and configured to detect physical contact with the recipient's tongue, electrical impedance changes due to movement of the recipient's tongue, and/or acoustic resonance changes within the recipient's mouth due to movement of the recipient's tongue.

15. The apparatus of claim 14, wherein the at least one sensor comprises at least one electrode in or on the recipient's tongue, and/or a front of the recipient's mouth, and/or the roof of the recipient's mouth, and/or back of the recipient's throat.

16. The apparatus of claim 8, wherein the at least one sensor is configured to be repeatedly inserted within the recipient's mouth and removed from the recipient's mouth.

17. The apparatus of claim 8, wherein the at least one sensor is configured to be worn outside the recipient's mouth.

18. The apparatus of claim 8, wherein the at least one sensor comprises at least one ultrasound detector configured to detect sound changes and/or acoustic resonance changes within the recipient's mouth and/or nose due to movement of the recipient's tongue.

19. The apparatus of claim 18, further comprising an ultrasonic source configured to generate ultrasonic signals to be detected by the at least one ultrasound detector.

20. An apparatus comprising:
at least one electrode configured to stimulate a portion of a recipient's body such that the portion, in response to being stimulated, moves from a first position to a second position;
at least one sensor configured to generate sensor signals indicative of positions of the portion; and
at least one controller configured to respond to the sensor signals by generating and transmitting control signals to the at least one electrode.

21. The apparatus of claim 20, wherein the at least one electrode is configured to be worn externally on the recipient's body.

22. The apparatus of claim 20, wherein the at least one sensor is responsive to a position of at least one device worn on the portion.

23. The apparatus of claim 20, wherein the at least one sensor is configured to be disposed in the recipient's body.

24. The apparatus of claim 22, wherein the at least one sensor is part of an appliance configured to be worn inside the recipient's mouth and the portion of the recipient's body comprises a portion of the recipient's tongue.
